(19) United States
(12) Patent Application Publication
(10) Pub. No.: US 2022/0044798 A1
(43) Pub. Date: Feb. 10, 2022

(54) AUDIO AND VIDEO PROCESSING FOR SAFETY ALERTS DURING AN IMAGING EXAMINATION

(71) Applicant: KONINKLIJKE PHILIPS N.V., Eindhoven (NL)

(72) Inventors: Olga Starobinets, Newtown, MA (US); Ranjith Naveen Tellis, Tewksbury, MA (US); Sandeep Madhukar Dalal, Winchester, MA (US); Hareesh Chamarthi, Cambridge, MA (US); Yuechen Madhukar Qian, Lexington, MA (US); Vijay Parthasarathy, Lexington, MA (US)

(21) Appl. No.: 17/392,692

(22) Filed: Aug. 3, 2021

Related U.S. Application Data
(60) Provisional application No. 63/061,829, filed on Aug. 6, 2020.

(51) Int. Cl. G16H 40/63; G16H 30/20

(52) U.S. Cl. CPC G16H 40/63; G16H 30/20

(57) ABSTRACT

A non-transitory computer readable medium (26) stores instructions executable by at least one electronic processor (20) to perform a method (100) of providing assistance from a remote expert (RE) to a local operator (LO) of a medical imaging device (2) during a medical imaging examination. The method includes: providing a user interface (UI) (29), on a remote workstation (12) operable by the remote expert, displaying a feed (17, 18) of at least an imaging bay (3) where the medical imaging device is disposed; detecting, by analysis of the feed, an event occurring in the imaging bay; and outputting, via the remote workstation, an alert (30) indicating the detected event.

This application claims the benefit of U.S. Provisional Application No. 63/061,829, filed on 6 Aug. 2020. This application is hereby incorporated by reference herein.

The following relates generally to the imaging arts, remote imaging assistance arts, remote imaging examination monitoring arts, and related arts.

### BACKGROUND

Medical imaging, such as computed tomography (CT) imaging, magnetic resonance imaging (MRI), positron emission tomography (PET) imaging, fluoroscopy imaging, and so forth, is a critical component of providing medical care, and is used in a wide range of medical fields, such as cardiology, oncology, neurology, orthopedics, to name a few. The operator of the medical imaging device used to acquire the medical images is typically a trained technologist, while interpretation of the medical images is often handled by a medical specialist such as a radiologist. Interpretation of radiology reports or findings by the radiologist can be handled by the patient's general practitioner (GP) physician or a medical specialist such as a cardiologist, oncologist, orthopedic surgeon, or so forth.

Currently, diagnostic imaging is in high demand. As the world population ages, the demand for quick, safe, high quality imaging will only continue to grow, putting further pressure on imaging centers and their staff. Under such conditions, errors are unavoidable, but can be often costly. One approach for imaging centers to boost efficiency and grow operations at no extra labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas (or from other sources, such as sensors (including radar sensors), console video feeds, microphones connected to Internet of Things (IoT) device, and so forth. In addition, these sources can be supplemented by other data sources like Health-Level 7 (HL7), Digital Imaging and Communications in Medicine (DICOM), Electronic Health Record (EHR) databases, and so forth.

The remote technologist (i.e. “super-tech”) is expected to be concurrently assigned to assist a number of different imaging bays at different sites that may be spread out across different cities or different states. In practice, however, the super-tech can only be paying attention to a single imaging bay at any given time. The super-tech will typically be assisting local technologists who actively call for super-tech support. However, situations may arise in which the super-tech's assistance would be beneficial, but the local technologist is unaware of the need for super-tech assistance, or chooses not to call for such assistance.

The following discloses certain improvements to overcome these problems and others.

### SUMMARY

In one aspect, a non-transitory computer readable medium stores instructions executable by at least one electronic processor to perform a method of providing assistance from a remote expert to a local operator of a medical imaging device during a medical imaging examination. The method includes: providing a user interface (UI), on a remote workstation operable by the remote expert, displaying a feed of at least an imaging bay where the medical imaging device is disposed; detecting, by analysis of the feed, an event occurring in the imaging bay; and outputting, via the remote workstation, an alert indicating the detected event.

In another aspect, an apparatus for providing assistance from a remote expert to a local operator during a medical imaging examination performed using a medical imaging device includes a workstation operable by the remote expert. At least one electronic processor is programmed to: provide a UI, on the workstation, displaying a feed of at least an imaging bay where the medical imaging device is disposed; track a workflow of the medical imaging examination; detect, by analysis of the feed and analysis of the workflow, an event occurring in the imaging bay; and output, via the remote workstation, an alert indicating the detected event.

In another aspect, a method of providing assistance from a remote expert to a local operator during a medical imaging examination includes: providing a UI, on a remote workstation operable by the remote expert, displaying a plurality of feeds of a corresponding number of imaging bays at a corresponding number of medical sites where a corresponding number of medical imaging devices is disposed; detecting, from the plurality of feeds, an event occurring in at least one of the imaging bays; and outputting, via the remote workstation, an alert indicating the detected event.

One advantage resides in providing a remote expert or radiologist assisting a technologist in conducting a medical imaging examination with positional awareness of local imaging examination(s) which facilitates providing effective assistance to one or more local operators at different facilities.

Another advantage resides in improving efficiency of assistance from a remote expert to one or more local operators by providing the remote expert with automated alerts calling attention to events occurring in imaging workflows being performed by the local operators that may suggest remote expert assistance would be beneficial.

Another advantage resides in providing alerts to a remote expert of events occurring during a procedure operable by a local operator.

Another advantage resides in providing a remote reviewer, such as a remote operator providing imaging examination support from a remote service center, or a remotely located radiologist providing image quality review during the imaging examination, with awareness of the medical imaging examinations which facilitates providing assistance to a one or more local operators at different facilities, and may enable the remote reviewer to provide preemptive corrective advice to the local imaging technologist.

Another advantage resides in avoiding delaying of imaging procedures based on incorrect examination settings.

Another advantage resides in increasing patient safety and comfort during a medical imaging examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

### DETAILED DESCRIPTION

The following discloses leveraging video feeds of a medical imaging device bay, and possibly other input such as an audio feed provided by a microphone, to automatically detect situations in which super-tech assistance may be beneficial and to provide alerts to the super-tech (and possibly also to the local technologist in the medical imaging device bay) in such cases.

The alerting approach utilizes video feeds provided from the imaging bays to which the super-tech is assigned. Microphones are also contemplated to be provided in the imaging bays to provide audio feeds. Video and/or audio feeds may also be provided from other areas, such as the waiting area.

The various video and audio feeds are automatically processed to detect situations in which super-tech assistance may be beneficial, based on detected features such as locations of individuals in the video feeds and their clothing (from which the patient, imaging technologist, nurse, or other roles are identified), detection of relevant hardware (e.g. imaging coils) being used (or not being used) in the imaging examination, patient movements of various types, loud voices in the audio feed, verbal calls for assistance in the audio feed, and so forth.

Additionally, the alerting system preferably tracks the workflow. This may be done based on analysis of the video feeds (e.g., detecting whether the patient is in the bore and therefore in the imaging stage) and timestamps or sequences of such detected features (e.g., if the patient is currently outside the bore and was previously inside the bore then the workflow stage is likely to be the patient unloading stage; whereas, if the patient is currently outside the bore and has not previously been inside the bore then the workflow stage is likely to be the patient loading stage. The alerting system also preferably has information about the specific imaging examination being performed, for example obtained from information contained in an electronic schedule of imaging examinations assigned to the super-tech.

The alerts are then identified based on the detected video and audio features and the workflow tracking. Alerts are typically sent to the remote medical expert or super-tech, and may also be sent to the local technologist. Optionally, alerts may be rated based on severity and the manner of the alerts chosen based on the severity. For example, an alert rated as critical may be issued as an audio alarm in combination with a flashing textual alert; whereas, an alert rated as low priority may be issued as a pop-up window or other less obtrusive notification shown on the super-tech's workstation.

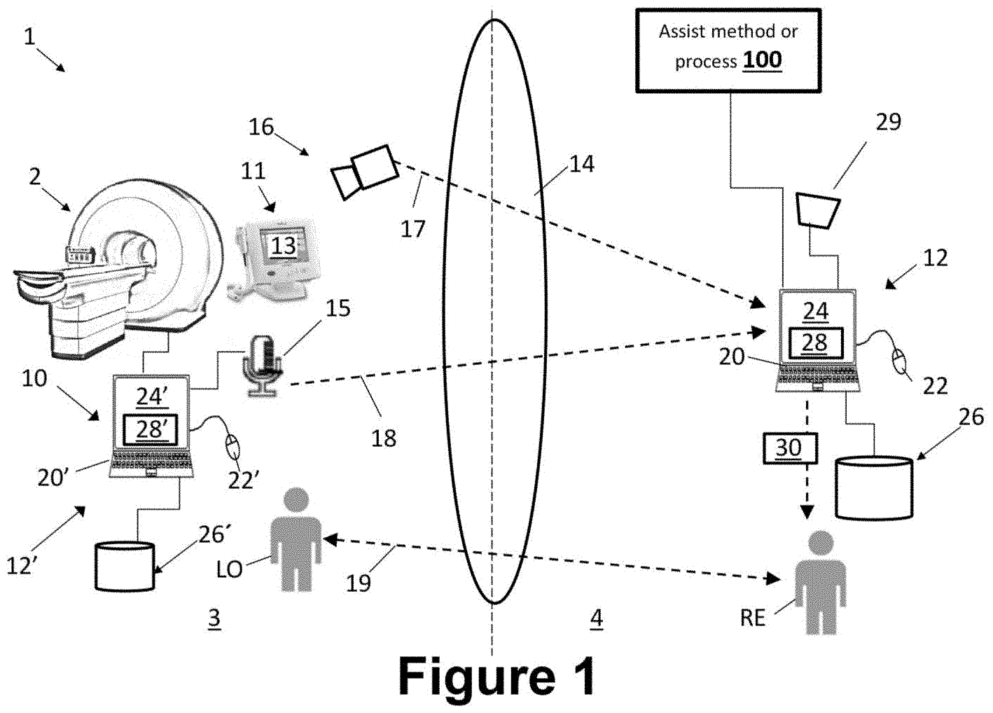

With reference to FIG. 1, an apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator controls the medical imaging device 2 via an imaging device controller 10. The remote operator is stationed at a remote workstation 12 (or, more generally, an electronic controller 12).

To provide for contrast-enhanced imaging, a contrast injector 11 is configured to inject the patient with a contrast agent. The contrast injector 11 is a configurable automated contrast injector having a display 13. The user (usually the imaging technologist) loads a vial or syringe of contrast agent (or two, or more, vials of different contrast agent components) into the contrast injector 11, and configures the contrast injector 11 by entering contrast injector settings such as flow rates, volumes, time delays, injection time durations, and/or so forth via a user interface (UI) of the contrast injector 11. The UI may be a touch-sensitive overlay of the display 13, and/or physical buttons, keypad, and/or so forth. In a variant embodiment, the contrast injector 11 is integrated with the imaging device controller 10 (e.g., via a wired or wireless data connection), and the contrast injector 11 is controlled via the imaging device controller 10, including displaying the contrast injector settings in a (optionally selectable) window on the display of the imaging device controller 10. In such an embodiment, the dedicated physical injector display 13 of the contrast injector may optionally be omitted (or, alternatively, the dedicated physical injector display 13 may be retained and the contrast settings displayed at both the dedicated physical injector display 13 and at the imaging device controller 10). In general, the automated contrast injector 11 can employ any suitable mechanical configuration for delivery of the contrast agent (or agents), such as being a syringe injector, a dual-syringe injector, pump-driven injector, or so forth, and may include hardware for performing advanced functions such as saline dilution of the contrast agent, priming and/or flushing of the contrast injection line with saline, and/or so forth.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote expert RE and local operator LO ends for electronic data communications. In addition, while FIG. 1 shows a single remote service center 4, it will be appreciated that the medical imaging device bays 3 is in communication with multiple medical bays via the communication link 14.

As diagrammatically shown in FIG. 1, in some embodiments, a camera 16 (e.g., a video camera) is arranged to acquire a video stream or feed 17 of a portion of a workspace of the medical imaging device bay 3 that includes at least the area of the imaging device 2 where the local operator LO interacts with the patient, and optionally may further include the imaging device controller 10. In other embodiments, a microphone 15 is arranged to acquire an audio stream or feed 18 of the workspace that includes audio noises occurring within the medical imaging device bay 3 (e.g., verbal instructions by the local operator LO, questions from the patient, and so forth). The video stream 17 and/or the audio stream 18 is sent to the remote workstation 12 via the communication link 14, e.g. as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or FaceTime or other screen-sharing program), or an audio-video call.

FIG. 1 also shows, in the remote service center 4 including the remote workstation 12, such as an electronic processing device, a workstation computer, or more generally a computer, which is operatively connected to receive and present the video feed 17 of the medical imaging device bay 3 from the camera 16 and/or to the audio feed 18. Additionally or alternatively, the remote workstation 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the remote workstation 12 via a loudspeaker 29. In some examples, the audio feed 18 can be an audio component of an audio/video feed (such as, for example, recording as a video cassette recorder (VCR) device would operate).

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote workstation 12. The ROCC may be housed in a single physical location, or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies.

The medical imaging device controller 10 in the medical imaging device bay 3 also includes similar components as the remote workstation 12 disposed in the remote service center 4. Except as otherwise indicated herein, features of the medical imaging device controller 10, which includes a local workstation 12′, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the remote workstation display 24 at the remote location 4.

Furthermore, as disclosed herein, the remote workstation 12 performs a method or process 100 for assisting local operators LO of respective medical imaging devices 2 during medical imaging examinations by a remote expert RE. The instructions to perform the method 100 are stored in the non-transitory computer readable medium 26 of the remote workstation 12.

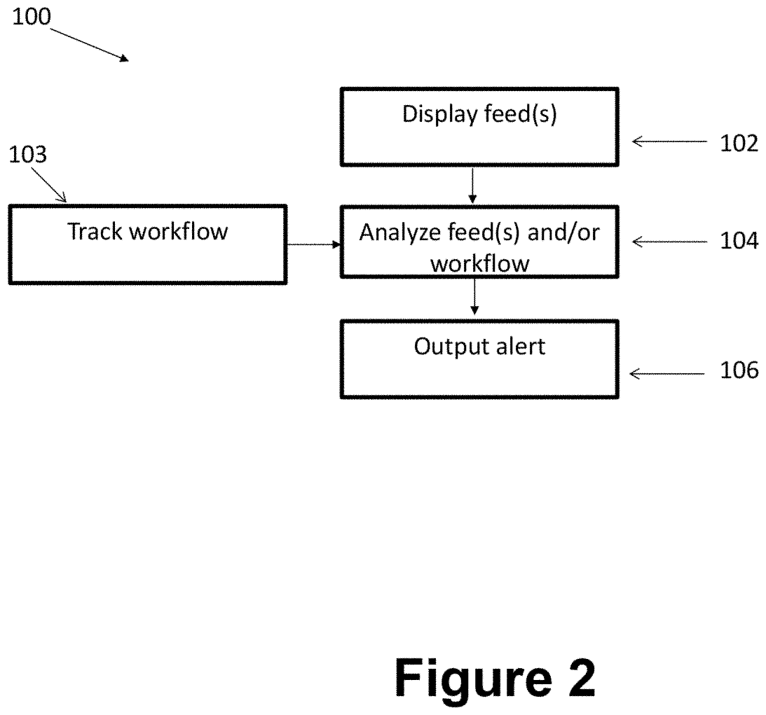

With reference to FIG. 2, and with continuing reference to FIG. 1, an illustrative embodiment of the method 100 is diagrammatically shown as a flowchart. To begin the method 100, an imaging examination is commenced by the local operator LO using the medical imaging device 2. An event can occur during the examination which requires assistance from a remote expert RE. The video feed 17 (acquired by the one or more cameras 16 and/or the audio feed 18 (acquired by the one or more microphones 15) are routed to the remote workstation 12 for analysis.

At an operation 102, as the feed(s) 17, 18 is/are received at the ROCC center on the remote workstation 12, the GUI 28 is provided on the display device 24 of the remote workstation 12 to display the feed(s) 17, 18. In some examples, the feed(s) 17, 18 can include a plurality of feeds at a corresponding number of imaging bays 3. At an optional operation 103, a workflow of the medical imaging examination is tracked. At an operation 104, an event occurring the medical imaging bay 3 is detected based on analysis of the feed(s) 17, 18 (and based on analysis of the tracked workflow where applicable). At an operation 106, an alert 30 is output on the remote workstation 12 indicating the detected event. In some examples, the alert 30 can also be output to the local operator LO.

In some embodiments, operation 104 includes detecting an event based on the analysis of the feed(s) 17, 18 and the analysis of the workflow provided by the tracking operation 103. In one example, the workflow tracking 103 includes analyzing the feed(s) 17, 18 to track the events in the workflow, and the detected event can be a workflow event of the workflow (e.g., the contrast injector 11 activating is an event of the workflow). In another example, the detecting of the event includes analyzing timestamps of the tracked workflow to detect the event.

In other embodiments, the feed 17, 18 includes at the least the video feed 17 acquired by the camera 16 of the imaging bay 3. In one example, the detected event includes a location or position of an individual (e.g., the local operator LO, technologist aides, the patient, and so forth) in the video feed 17 by analysis of the video feed, and the corresponding alert 30 indicates that the location or position of the individual is incorrect. In some examples, to protect patient privacy, instead of conveying a live video feed 17 of the imaging bay 3, the video feed can be processed and individuals can be replaced by abstract figures in the video feed, with their appropriate roles marked (e.g. color-coded) based on their appearance and actions.

In one example, the video feed 17 is analyzed to confirm a correct positioning of the patient within the medical imaging device 2. To do so, the patient's position can be monitored in real time, and contrasted with the requirements dictated by the imaging workflow (e.g., headfirst vs. feet first, etc.). If the patient is positioned incorrectly, a corresponding alert 30 can be output.

In another example, the video feed 17 is analyzed to detect whether the patient is wearing, for example, protective headphones (not shown), and has an alert ball (not shown) in hand. (An alert ball is a handheld alert button, squeezable ball, or the like which a patient loaded into an Mill bore or other constricted imaging examination space can press, squeeze, or otherwise easily activate to generate an alert calling for assistance). A corresponding alert 30 can be output if the medical imaging examination is about to start and the patient is not wearing the protective headphones or does not have the alert ball in hand.

In another example, an alert 30 can be output if the patient is not wearing appropriate clothing compatible with the medical imaging device 2 (e.g., leisure wear vs. a hospital gown; or, a watch or other metal-containing wearable may be detected in the video, which is not permitted in an Mill examination).

In yet another example, the video feed 17 of an Mill examination is analyzed to detect whether the patient's hands are clasped. This can be a problem because time-varying magnetic fields generate electrical currents, and when the patient's hands are clasped this can form a closed electrical conduction loop, which can lead to excessive peripheral nerve stimulation. Hence, a corresponding alert 30 can be output if patient's hands are detected to be clasped based on video feed analysis.

In other examples, the detected event includes movement of a patient during an imaging examination detected by analysis of the video feed 17, and the corresponding alert 30 indicates patient movement. In one example, the detected event includes whether patient is attempting to get up (or otherwise move) from during the medical imaging examination. A corresponding alert 30 can be output to indicated movement, and also may include an indication to review the images and potentially restart the examination if the images are unusable due to the detected patient motion.

In further embodiments, the detected event includes an issue with either the medical imaging device 2 itself, or with a workflow step of the medical imaging device. In one example, the detected event comprises a misplacement of a hardware component of the imaging device 2 detected by analysis of the video feed 17, and the corresponding alert 30 indicates the hardware component is misplaced. In other examples, the detected event can include malfunction hardware components, difficulty with positioning or attaching hardware components, or with workflow steps (e.g., how long it takes to position the patient on the medical imaging device 2, how long it takes to clean the room following the examination, a time stamp for when the contrast is being injected, etc.). A corresponding alert 30 can then be output.

In other embodiments, the feed 17, 18 includes at the least the audio feed 18 acquired by the microphone 15 of the medical imaging device controller 10. In one example, the detected event comprises a malfunction of a hardware component of the imaging device 2 detected by analysis of the audio feed 18 (e.g., detected as excessive vibrational noise or the like), and the corresponding alert 30 indicates the hardware component is malfunctioning. In another example, the audio feed 18 is analyzed to troubleshoot the medical imaging device 2, and a corresponding alert 30 is output. In a further example, the audio feed 18 is analyzed to detect whether the patient has squeezed an alarm ball, and a corresponding alert 30 can be output.

In further embodiments, the detected event includes a verbal noise, such as a call for help or assistance from the local operator LO, such as for assistance with a workflow step or to address a combative situation. A corresponding alert 30 can then be output. This could be done based on total audio energy in the 20-20,000 Hz range (or some subset thereof), and/or by applying speech recognition to the audio to extract semantic textual content that may trigger an alert, such as detecting the word or phrase “help” or “need assistance”. In another example, the audio feed 18 can be analyzed to determine one or more baseline audio characteristics. The detected event comprises unusual audio in the audio feed 18 detected by comparing the audio feed with the baseline audio characteristics. A corresponding alert can then be output to indicate that unusual audio (e.g., audio intensity above the baseline intensity, or audio frequency higher than baseline possibly indicating a person speaking in an elevated pitch) has been detected.

In other examples, the remote expert RE can mute the loudspeaker 29 of the remote workstation 12 (e.g., with a mute input) so that the audio feed 18 is not output, which may avoid an unnecessary distraction. As a result, once the audio feed 18 is analyzed, the alert 30 can be output to include an alert on the display device 24 of the remote workstation 12 to have the remote expert RE unmute the loudspeaker 29 to receive the audio feed 18 (or the remote workstation can be programmed to automatically unmute the loudspeaker when such an alert is output, or a flashing light or beep can be output to have the remote expert unmute the loudspeaker). This can be performed by providing a button on the GUI 28 for the patient to select with the at least one user input device 22 to unmute the loudspeaker 29.

In some embodiments, the alert output operation 106 can include determining a significance of the alert 30. For example, if multiple alerts 30 are output (e.g., one or more alerts output on the display device 24 of the remote workstation 12, and/or one or more alerts output via the loudspeaker 29), then the remote workstation 12 can analyze these alerts to determine a significance of the alerts. The alerts 30 can be ranked relative to each other, or the alerts can be ranked according to a predetermined significance threshold. For example, alerts that pertain to patient safety may be ranked higher than alerts that pertain to image quality. Alerts 30 having a higher significance can be output first to the remote expert RE.

In other embodiments, the alert output operation 106 can include correlating a response time to the alerts 30. The remote workstation 12 can analyze the alerts 30 to determine whether any of the alerts 30 need to be resolved more quickly than other output alerts. For example, during initial patient preparation, an alert 30 pertaining to the patient being positioned incorrectly may initially produce only an alert in the form of a text message. However, if the workflow is transitioning to the patient loading phase, then this alert 30 may be escalated, e.g. output as an audible warning that the patient is not correctly positioned. An alert 30 due to detected patient motion during an imaging sequence may not be initially presented at all, but only presented at the end of the imaging sequence as the technician is about to review the acquired images. This avoids distracting the local operator LO during image acquisition. As yet another example, detection that the patient's hands are clasped during MRI examination setup may produce a textual alert 30; whereas, detection that the patient's hands are clasped during MM image acquisition when the patient is being subjected to strong electromagnetic fields (thus creating an immediate patient safety concern) may result in an immediate audible alert to unclasp the hands. To do such analyses, a response time can be correlated to each output alert 30. In some examples, an alert 30 with a response time approaching expiration (e.g., mispositioned patient about to be loaded into the imaging device; or, patient with hands clasped as the imaging acquisition is about to be started; et cetera) can be escalated to the remote expert RE (e.g., by displaying a message on the display device 24, repeating the alert via the loudspeaker 29, and so forth). In other examples, if the remote expert RE who received the alert 30 cannot resolve the alert before expiration of the response time, the remote workstation 12 can transfer the alert 30 to an available remote expert for resolving.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of providing assistance from a remote expert (RE) to a local operator (LO) of a medical imaging device during a medical imaging examination, the method comprising:
providing a user interface (UI), on a remote workstation operable by the remote expert, displaying a feed of at least an imaging bay where the medical imaging device is disposed;
detecting, by analysis of the feed, an event occurring in the imaging bay; and
outputting, via the remote workstation, an alert indicating the detected event.

2. The non-transitory computer readable medium of claim 1, wherein the feed comprises a video feed of at the least the imaging bay.

3. The non-transitory computer readable medium of claim 2, wherein:
the detected event comprises a location or position of an individual in the video feed detected by analysis of the video feed; and
the alert indicates the location or position of the individual is incorrect.

4. The non-transitory computer readable medium of claim 2, wherein:
the detected event comprises a misplacement of a hardware component of the imaging device detected by analysis of the video feed; and
the alert indicates the hardware component is misplaced.

5. The non-transitory computer readable medium of claim 2, wherein:
the detected event comprises a movement of a patient during an imaging examination detected by analysis of the video feed; and
the alert indicates patient movement.

6. The non-transitory computer readable medium of claim 1, wherein the feed comprises an audio feed of at the least the imaging bay.

7. The non-transitory computer readable medium of claim 6, wherein:
the detected event comprises a malfunction of a hardware component of the imaging device detected by analysis of the audio feed; and
the alert indicates the hardware component is malfunctioning.

8. The non-transitory computer readable medium of claim 6, wherein the method further comprises:
to determine one or more baseline audio characteristics of the audio feed;
wherein the detected event comprises unusual audio in the audio feed detected by comparing the audio feed with the baseline audio characteristics; and
wherein the alert indicates unusual audio has been detected.

9. The non-transitory computer readable medium of claim 7, wherein:
the remote workstation includes a loudspeaker configured to output the audio feed and further has a mute input whereby the remote expert (RE) can select to mute the output of the audio feed; and
in the case of the audio feed being muted, the alert indicating unusual audio has been detected comprises turning off the mute whereby the loudspeaker outputs the audio feed.

10. The non-transitory computer readable medium of claim 6, wherein:
the detected event comprises a verbal call for assistance detected by analysis of the audio feed; and
the alert indicates a request for assistance.

11. The non-transitory computer readable medium of claim 1, wherein the method further comprises:
tracking a workflow of the imaging examination;
wherein the detecting of an event is based on analysis of the feed and the analysis of the workflow.

12. The non-transitory computer readable medium of claim 11, wherein the tracking of the workflow includes:
analyzing the feed to track the events in the workflow.

13. The non-transitory computer readable medium of claim 11, wherein the detecting of an event based on analysis of the feed and the analysis of the workflow includes:
analyzing timestamps of the tracked workflow.

14. The non-transitory computer readable medium of claim 11, wherein the detected event is a workflow event of the tracked workflow.

15. The non-transitory computer readable medium of claim 1, wherein the feed comprises a plurality of feeds at a corresponding number of imaging bays.

16. The non-transitory computer readable medium of claim 1, wherein the outputting includes:
outputting the alert to the local operator (LO).

17. The non-transitory computer readable medium of claim 1, wherein the outputting includes:
determining a significance of the alert.

18. The non-transitory computer readable medium of claim 1, wherein the outputting includes:
correlating a response time to the alert.

19. The non-transitory computer readable medium of claim 18, wherein the outputting includes:
transferring the alert to another remote expert (RE) when the remote expert who received the alert cannot resolve the alert before expiration of the response time.

20. An apparatus for providing assistance from a remote expert (RE) to a local operator (LO) during a medical imaging examination performed using a medical imaging device, the apparatus comprising:
a workstation operable by the remote expert; and
at least one electronic processor programmed to:
provide a user interface (UI) on the workstation displaying a feed of at least an imaging bay where the medical imaging device is disposed;
track a workflow of the medical imaging examination;
detect, by analysis of the feed and analysis of the workflow, an event occurring in the imaging bay; and
output, via the remote workstation, an alert indicating the detected event.

21. The apparatus of claim 20, wherein the feed comprises one of a video feed or an audio feed of at the least the imaging bay.

22. The apparatus of claim 20, wherein the at least one electronic processor is further programmed to:
output the alert to the local operator (LO).

23. A method of providing assistance from a remote expert (RE) to a local operator (LO) during a medical imaging examination, the method comprising:
providing a user interface (UI), on a remote workstation operable by the remote expert, displaying a plurality of feeds of a corresponding number of imaging bays at a corresponding number of medical sites where a corresponding number of medical imaging devices is disposed;
detecting, from the plurality of feeds, an event occurring in at least one of the imaging bays; and
outputting, via the remote workstation, an alert indicating the detected event.
